(19) United States
(12) Patent Application Publication
(10) Pub. No.: US 2002/0133832 A1
(43) Pub. Date: Sep. 19, 2002

(54) Model systems for neuordegenerative and cardiovascular disorders

(76) Inventors: Dianne M. Perez, Broadview Heights, OH (US); Michael J. Zuscik, Westlake, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/052,589

(22) Filed: Jan. 18, 2002

Related U.S. Application Data
(62) Division of application No. 09/568,255, filed on May 10, 2000.

(51) Int. Cl. A01K 67/00; A01K 67/027

(52) U.S. Cl. 800/9; 800/18

(57) ABSTRACT

New tools for determining the role the α_{1B} adrenergic receptor plays in the physiology and pathology of the brain and the cardiovascular system are provided The tools are transgenic non-human mammalian animals, particularly transgenic mice, that have integrated into the genomes of their somatic cells a transgene encoding an exogenous, wild-type α1B adrenergic receptor or a variant thereof. The transgenic animals of the present invention exhibit phenotypical symptoms similar to those exhibited by individuals with neurodegenerative diseases, particularly Parkinson's disease or epilepsy. Such mammals also exhibit phenotypical symptoms similar to individuals with cardiovascular diseases such as hypertrophy of the heart and hypotension. Accordingly, these transgenic mammals are also useful for screening for drugs that ameliorate these cardiovascular conditions. Also provided is a method of determining the ability of a test agent or compound to modulate or block function of the α_{1B} adrenergic receptor. The method comprises administering the test agent to a transgenic non-human animal which is expressing a constitutively active form of the α_{1B} receptor, or elevated levels of the wild-type α_{1B} receptor on the cell surface of various organs, and then assaying for changes in α_{1B} receptor function. The present invention also relates to methods for treating neurodegenerative disorders in a subject, particularly neurodegenerative disorders evidenced by abnormal locomoter activity or seizures. In one embodiment, the method comprises administering a pharmaceutical composition comprising a biologically effective amount of an α₁ adrenergic receptor antagonist to the subject.

### BACKGROUND

The adrenergic receptor family is a group of heptahelical G protein-coupled receptors that mediate the effects of the sympathetic nervous system. At present, this family is known to contain three α1, three α2, and three B receptor subtypes. All of the receptors in this family bind to and are activated by the hormones epinephrine and norepinephrine. By a series of steps involving G proteins, the activated receptors then activate an effector. In the case of the α_{1A} adrenergic receptors the effector is phospholipase C; in the case of the α₂ and β, the effector is adenylate cyclase.

Cells expressing α₁-adrenergic receptors are found in the heart, liver, kidney, brain and spleen. Surprisingly, such cells do not express a single subtype. Indeed, in the brain, all three α1 subtypes co-exist on a single cell. Attempts have been made to elucidate the specific role each α1 receptor plays in the physiology and pathophysiology of such cells using agonists or antagonists which bind with greater affinity to one of the oil receptors. However, the antagonists that are currently available do not have sufficient selectivity to discriminate between the subtypes. Moreover, such studies typically involve a single bolus injection of the respective agonist or antagonist, and, therefore, cannot identify the pathologies that result from chronic activation of a single receptor subtype.

Accordingly, it is desirable to have new tools and methods which can be used to determine the effect of chronic activation of a single α₁ adrenergic receptor subtype. A tool which can be used to screen for antagonists for a particular α₁ adrenergic receptor and to determine the systemic side effects of such antagonists is especially desirable.

### SUMMARY OF THE INVENTION

The present invention provides new tools for determining the role the α_{1B} adrenergic receptor plays in the physiology and pathology of the brain, cardiovascular system and virtually all organs that express the α_{1B} subtype. The tools are transgenic non-human mammalian animals, particularly transgenic mice, that have integrated into the genomes of their somatic and germline cells a transgene encoding an exogenous, wild-type α1B adrenergic receptor or a variant thereof. As compared to normal non-transgenic mice, the transgenic animals whose genomes comprise a transgene encoding an exogenous wild-type α1B adrenergic receptor have elevated levels of the α_{1B} receptor on the cell surface. The transgenic animals whose genomes comprise a transgene encoding a variant α1B adrenergic receptor have a constitutively active on the cell surface. In one embodiment, the transgene encodes a variant form of the hamster α1B adrenergic receptor in which the cysteine at position 128 in the amino acid sequence of the wild-type receptor is replaced with a phenylalanine. In another embodiment, the transgene encodes a variant form of the hamster α1B adrenergic receptor in which the cysteine at position 128 is replaced with a phenylalanine, the alanine at position 204 is replaced with a valine, and the alanine at position 293 is substituted with a glutamic acid. The transgenic animals of the present invention exhibit phenotypical symptoms similar to those exhibited by individuals with neurodegenerative diseases, particularly Parkinson's disease or epilepsy. Accordingly, these transgenic mammals are useful model systems for screening for drugs that ameliorate the symptoms of such neurodegenerative diseases. Such mammals also exhibit phenotypical symptoms similar to individuals with cardiovascular diseases such as hypertrophy of the heart and hypotension. Accordingly, these transgenic mammals are also useful for screening for drugs that ameliorate these cardiovascular conditions.

The present invention relates to a method of determining the ability of a test agent or compound to modulate or block function of the α_{1B} adrenergic receptor. A preferred method comprises administering the test agent to a transgenic non-human animal which is expressing a constitutively active form of the α_{1B} receptor, or elevated levels of the wild-type α_{1B} receptor on the cell surface of various organs, and then assaying for changes in α_{1B} receptor function. Such method is useful for identifying compounds which are able to ameliorate the symptoms that result from chronic activation of the α_{1B} adrenergic receptor and assessing the efficacy of the test compound on pathological symptoms that are associated with chronic activation of the α_{1B} adrenergic receptor.

The present invention also relates to methods for treating neurodegenerative disorders in a subject, particularly neurodegenerative disorders evidenced by abnormal locomoter activity or seizures. In one embodiment, the method comprises administering a pharmaceutical composition comprising a biologically effective amount of an oil adrenergic receptor antagonist to an animal. As used herein the term “α1 adrenergic antagonist” refers to compounds that bind selectively to the α1 adrenergic receptors and block signaling.

### BRIEF DESCRIPTION OF THE FIGURES

FIG. 1 shows the nucleotide sequence of the cDNA which encodes the hamster wild-type α_{1B} adrenergic receptor and the predicted amino acid sequence encoded by this nucleotide sequence.

FIG. 2 is the DNA sequence of the promoter of the murine alB adrenergic receptor.

FIG. 3 is a schematic representation of the method used to prepare a vector comprising a sequence encoding the alB adrenergic receptor.

FIG. 4. (A) A map of the trans gene construct showing the size of EcoRI fragments and the binding sites for α_{1B}- and SV40-specific southern probes. Three different transgenes were constructed with the only difference between each being the α_{1B}AR cDNA used (either the wild-type (WT), single mutant or triple mutant cDNA). (B) Southern blot analysis of genomic DNA from nontransgenic (NT)(−/−), heterozygous (+/−) and homozygous (+/+) W2 mice. Tail DNA samples were digested with EcoRl, run on 0.8% agarose gels, transferred to nitrocellulose and probed with either the α_{1B} probe or the SV40 probe. The OCIB probe hybridized to 3.0 and 1.6 kb fragments which represented the endogenous alBAR gene and the transgene respectively. Comparatively, the SV40 probe hybridized only to a 1.4 kb fragment which represented the transgene. (C) Bₘₐₓ determination was carried out via saturation binding in various α_{1B}AR -positive and -negative tissues using the α₁-antagonist 2-[β-(4-hydroxyl-3-[¹²⁵I]iodophenyl)ethylaminomethyl]tetralone ([¹²⁵I]HEAT) as the radioligand. Bₘₐₓ values in W2+/− mice that were significantly different from the corresponding non-transgenic (NT) values are labeled with an asterisk. Error bars represent SEM (N>5 for each tissue) and significance was determined using analysis of variance with a two-tailed Student's t test (p<0.05). (D) Inositol tri-phosphate (IP₃)levels. Error bars represent SEM (n=3 for each line) and significance was determined using analysis of variance with a two-tailed Student's t test (p<0.05). The asterisk (*) indicates significance from the NT group. The dagger (†) indicates significant increases compared to the W2+/− group. The double cross (‡) indicates significant increases compared to the S 1+/− group. (E) Hybridization pattern of the SV40 probe in a section cut from a NT mouse. (F) Hybridization pattern of the α_{1B} probe to endogenously expressed α_{1B}AR transcripts in a NT brain section. (G) Hybridization of the SV40 probe to message transcribed from the transgene in the brain of a W2+/− mouse. Cx=cortex; Rt=reticular thalamic nuclei; Hy=hypothalamus. (H) Transgene expression detected by the a1B probe. These positive regions coincide with regions identified in C and overlap the background expression of the endogenous gene.

FIG. 5. (A) The average litter size generated from homozygous parents was determined from a minimum of five mating pairs each for NT mice and all transgenic lines. Error bars represent SEM and significance was determined using analysis of variance with a two-tailed Student's t test (p<0.05). Average litter sizes that were significantly different from the average NT litter size are labeled with an asterisk (*). (B) S1+/−(●), T1+/−(▪) and T2+/−(▾) mice had reduced longevity compared to NT controls (▴). (C) At 14 months of age, W2+/+, S1+/−, T1+/− and T2+/− mice exhibited significantly lower body weights compared to NT controls. Error bars represent SEM (n>7 for each line) and significance was determined using analysis of variance with a two-tailed Student's t test (p<0.05). Transgenic mouse body weights that were significantly different from NT body weights are labeled with an asterisk (*).

FIG. 6. An Active Open Field Activity System (Harvard Apparatus, Holliston, Mass.) was used to monitor total activity, distance traveled/min and number of rearings/min in age matched NT (▴), W2+/−(▪), S1+/−(●) and T1+/−(▾) and T2+/−(♦) mice. The open field for these experiments was a 16 inch by 16 inch enclosure with infrared beams of light aimed to form grids near the floor and 3 inches above the floor of the enclosure. (A) Total activity, or the total number of beam breaks each minute, was determined in two month old mice for a total time of 15 min. (B) A computer algorithm was used to calculate the distance traveled/min during horizontal ambulation by mice of varying age. (C) The number of rearings/minute was determined by electronically tallying the number of times mice of varying age reared onto their hindlimbs, facilitating a greater than 1 sec beam break in the upper grid (3 inches above the floor of the enclosure). (D) Snapshot of an 11 month old S1+/− mouse showing elongation of the torso caused by sprawling and dragging of the hindlimbs. (E) The ability of terazosin and L-DOPA to rescue the reduced number of rearings/min seen in eighteen month old S1+/− and T1+/− mice was tested by administering a target dose of 0.05 mg terazosin/kg body weight/day via the drinking water. After a four week pretreatment with the drug, the number of rearings/min was determined. All error bars in the Figurere present SEM (n>3) and significance in D was determined using analysis of variance with a two-tailed Student's t test (p<0.05). Average rearings/min without terazosin that were significantly different from NT controls are labeled with an asterisk (*) Significant rescue of the rearing behavior by terazosin or L-DOPA in S1+/− and T1+/− mice is denoted with a dagger (†).

FIG. 7. (A) Sequence of seizure behaviors in a 12 month old T2+/− mouse. 1; behavioral arrest. 2; loss of balance and whole body jerking. 3; forelimb flexion. 4; recovery. (B) Comparison of percent seizure activity induced by open field stress in various lines of mice at 12 months of age. Seizure activity was quantitated by scoring a mouse as positive if it exhibited a grand mal-type seizure event at least once during a series of five daily exposures to the open field. Percent seizure activity was then calculated by dividing the number of seizure-positive mice by the total number of mice tested. The total number of mice tested for each case is shown above the respective column in the graph. The ability of terazosin to rescue T2+/− mice from the seizure phenotype was tested by administering a target dose of 0.05 mg/kg body weight/day via the drinking water. After a four week pretreatment with the drug, percent seizure activity was determined. (C) Identical experiment to that described in B, except percent seizure activity was determined in lines of mice at seven months of age that were exposed to intraperitoneal injection (IPI) stress. IPI stress was administered to the mice by intraperitoneal injection of 50 μt of sterile 0.9% NaCl.

FIG. 8. Hematoxylin/eosin (H&E) stains and tyrosine hydroxylase (TH) immuno-stains of 20 micron coronal brain sections cut through the forebrain of 10 or 11 month old NT, W2+/− and T2+/mice. (A) 100× view of an H&E stained 10 month old NT cortex. Arrowheads delineate the cortical laminae. (B) 100× view of an H&E stained age-matched W2+/− cortex. Arrowheads define the area displaying laminar disorganization. (C) 400× view of an area from the same NT cortex shown in A. (D) 400× view of an area from the same W2+/− cortex shown in B. Arrowheads identify cells displaying a morphology consistent with reactive astrocytes. Note the infiltration of these actrocytic cells relative to the section shown in C. (E) 400× view of an H&E stained 10 month old NT hypothalamic region. (F) 400× view of an H&E stained age-matched T2+/− hypothalamic region. Arrowheads identify cells displaying a morphology consistent with reactive astrocytes. Again note the infiltration of these astrocytic cells relative to the section shown in C. (G) 100× view of a region from an 11 month old NT brain encompassing the substantia nigra (SN) and the periaqueductal gray area (PAG). TH immuno-staining, using a 1:100 dilution of a sheep-anti-TH polyclonal antibody (Chemicon, Temecula, Calif.), is identified in the SN by arrowheads. (H) 100× view of an age matched T2+/− brain section encompassing similar areas as in G. Arrowheads identify TH immuno-staining. Note the reduced amount of TH immunoreactivity compared to the NT control shown in G. (I) 200× view of the substantia nigra from the same section shown in G. (J) 200× view of the substantia nigra from the same section shown in H. Again, note the reduced amount of TH immunoreactivity in T2+/− sections relative to the NT control shown in I. (K) 200× view of an H&E stained 11 month old NT brain section encompassing the peiraqueductal gray area. (L) 200× view of an H/E stained age-matched T2+/brain section showing an area analagous to the area shown in K. Arrowheads identify vacuolar spaces that have become prevalent in the T2+/− mice.

FIG. 9. Hematoxylin/eosin (H & E) of 20 micron coronal brain sections cut through the forebrain of 10 month NT, W2, or T2 mice. (A) 100 x view of an H&E stained 10 month old NT cortex. Arrowheads delineate the cortical laminae. (B) 100× view of an H&E stained age-matched W2+/− cortex. Arrowheads define the area displaying laminar disorganization. (C) 100× view of the T2 cortex of a mouse experiencing seizures. Arrows point to areas of vast neurodegeneration as evidenced by the dead space. (D) 100× view of the T2 hypothalamus in the same mouse as D. This section of the brain was also degenerativing as evidenced by the dead space (arrows).

FIG. 10. (A) Changes in basal blood pressure in NT, W2+/−, S1+/−, and T2+/− mice versus the time of recovery from the surgery. Mice 16-22 weeks of age, were weighed and anesthetized with a mixture of Ketaset-Acepromazine intraperitoneally. The neck and throat were shaved, then cleaned with Povidone-Iodine and 70% isopropyl alcohol. A surgical incisions was made in the throat area, and the right carotid artery was isolated. The distal end of the carotid artery was sealed off with suture while the proximal end was temporarily tied to facilitate the insertion of the the catheter through a nick in the artery. Once the catheter was inserted into place, sutures were tied around the carotid artery to prevent movement of the cathether, yet loosely to allow blood flow through the catheter. Then the catheter was tuneeled subcutaneously to the back of the neck and out of the body where another incision had been made. The arterial line was connected to a pressure transducer, and blood pressure readings were taken over seven hours. After mice were fully recovered from the anesthetic (8 hours), both the S1 and T2 mice displayed a significantly lower basal blood pressure than the WT or NT mice. (B) Blood pressure in anesthetized mice in response to pressure-induced changes caused by phenylephrine. Unconscious mice, 16-22 weeks of age, were cannulated via the femoral artery and changes in blood pressure recorded in response to the oca-adrenergic specific against, phenylephrine. S1 mice had significantly lower blood pressure in response to phenylephrine than NT controls (n>5).

FIG. 11 is a bar graph showing the heart to body weight ratio of non-transgenic (NT) W2+/−, S1+/−, and T2+/− mice at 16-22 weeks of age. Hearts were blotted 5 times on absorbant paper before measurement was made. The organ to body weight ratios of the liver, brain, lung, did not change.

FIG. 12 is a graph showing the plasma levels of total catecholamines in NT, W2+/−, S1 +/−, and T2+/− mice. Mice, 16-22 weeks of age, were anesthetized with Inactin and after 5 minutes of unconsciousness, blood was drawn via the vena cava and pooled with 4 other mice of the same line. Catecholamines were measured by a radioenzymatic assay method.

### DETAILED DESCRIPTION OF THE INVENTION

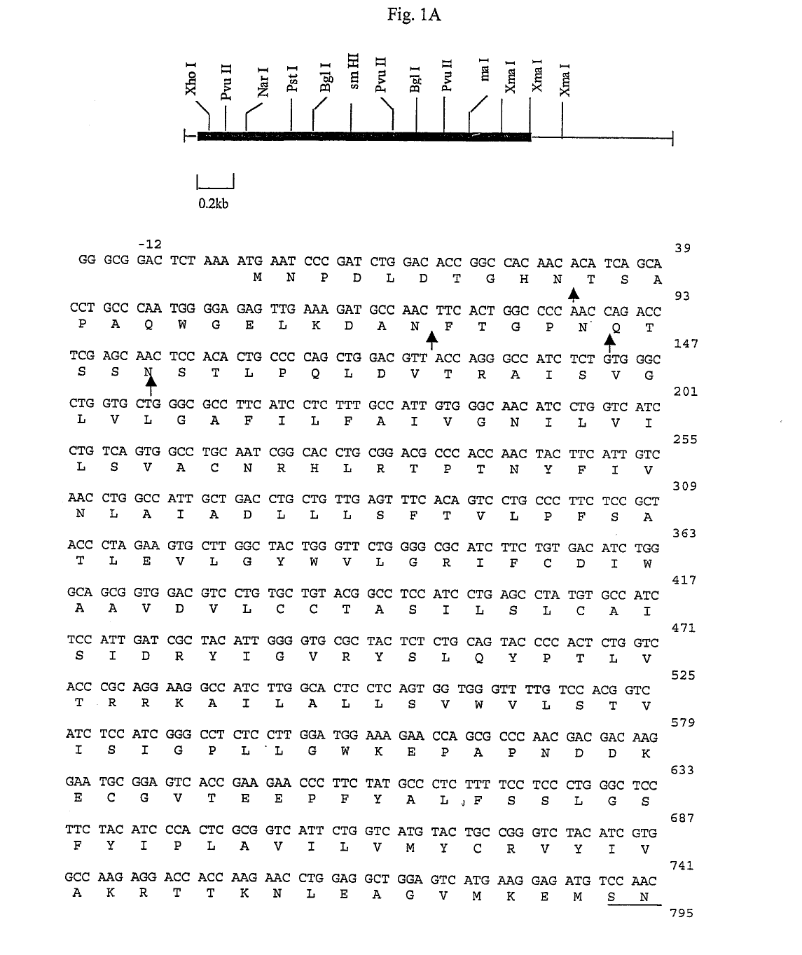

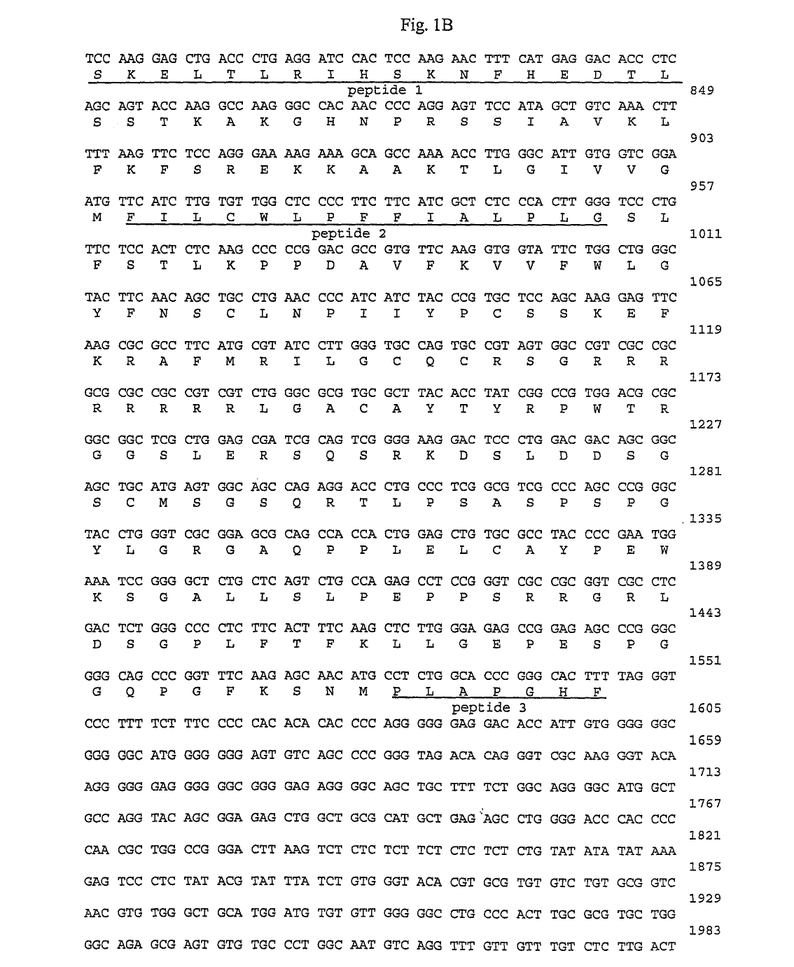

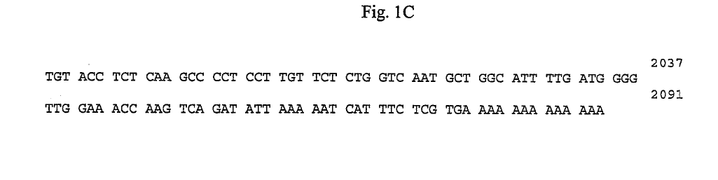

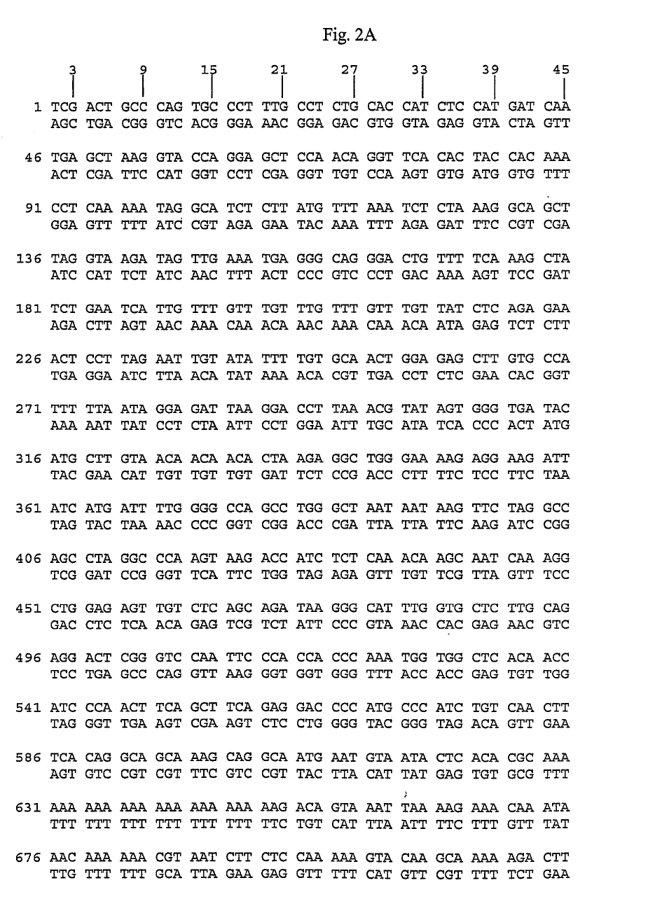

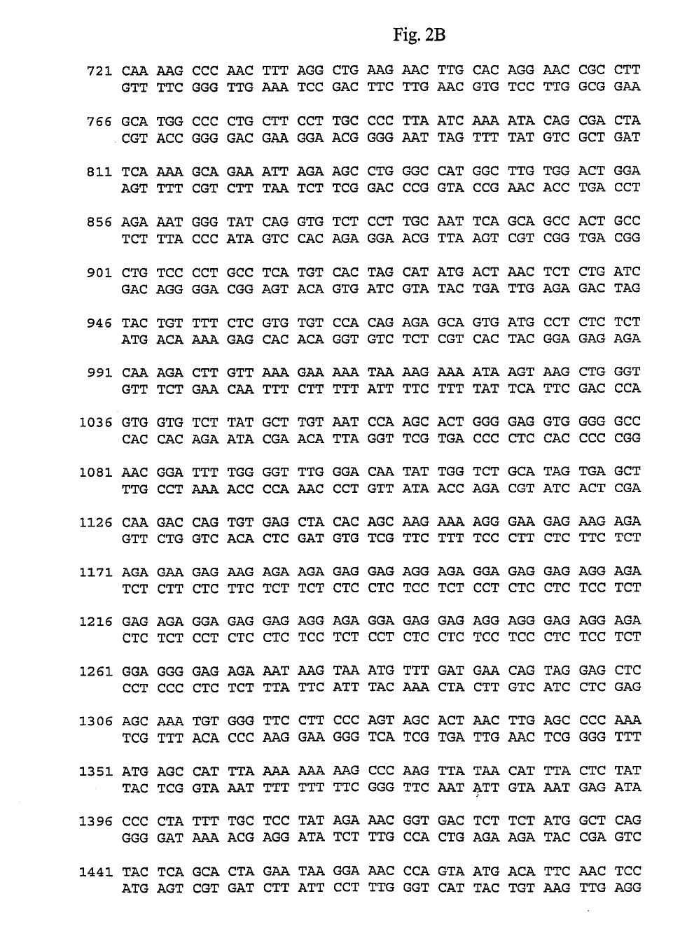

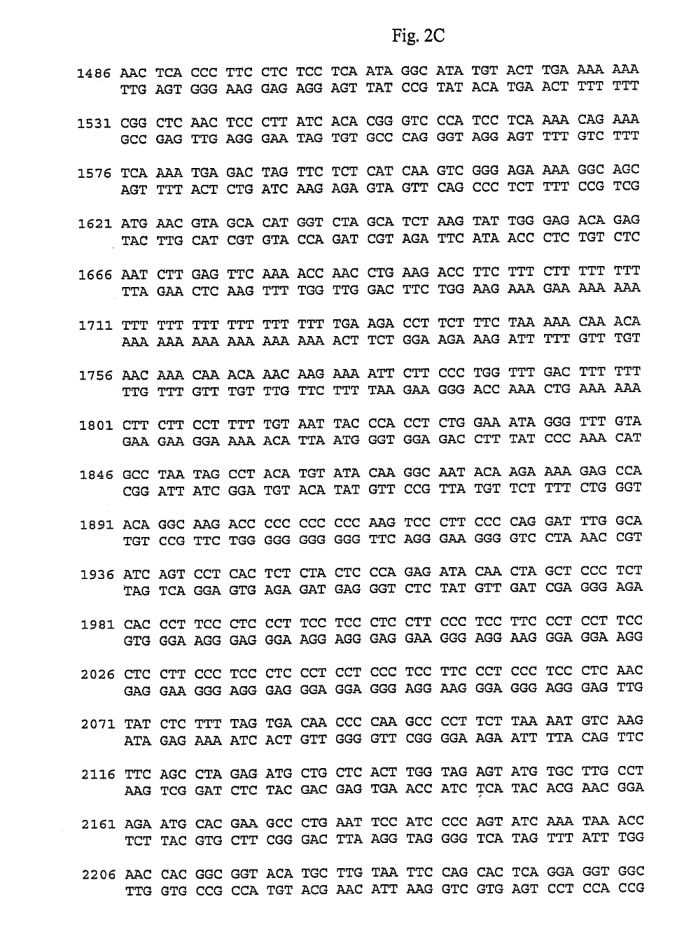

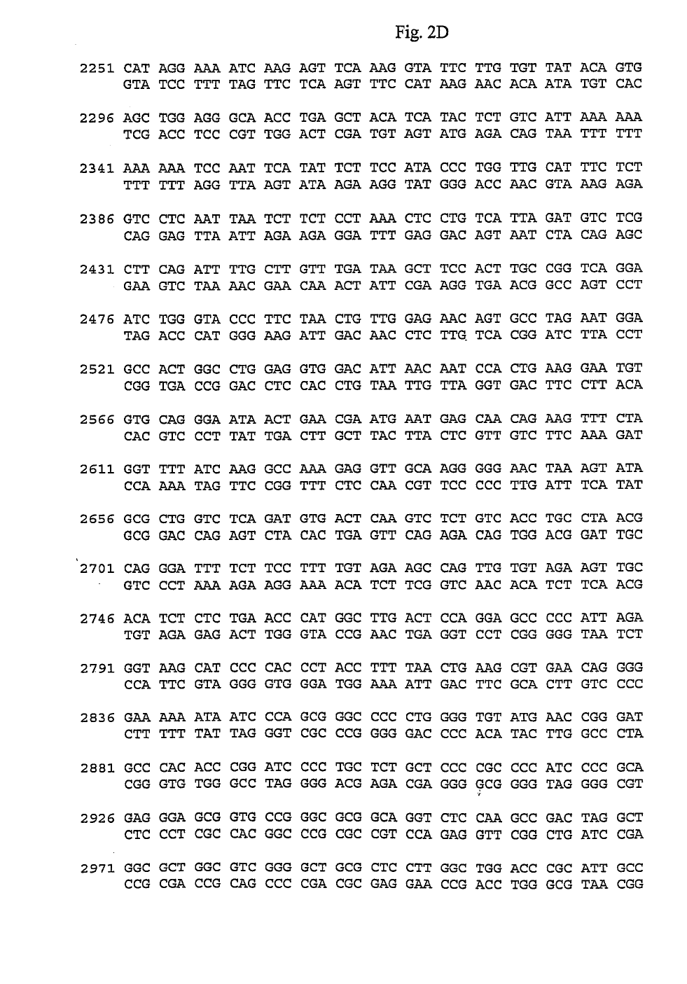

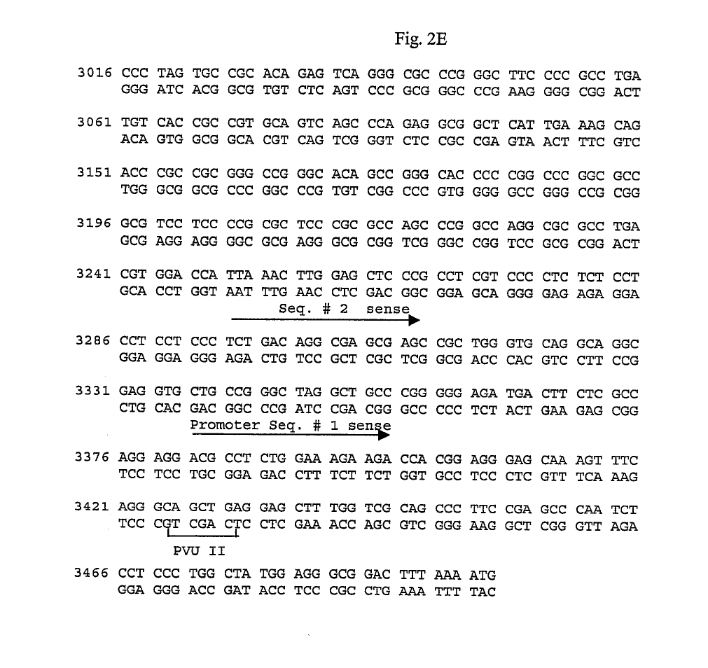

The present invention provides a tool for analyzing the molecular mechanism of the α_{1B} adrenergic receptor in the physiology and pathophysiology of individual organ systems or, collectively, in a whole animal. Such tool is a transgenic animal that has incorporated into its genome a nucleic acid encoding an exogenous wild type α_{1A}, α_{1B}, or α_{1D} adrenergic receptor or a variant thereof. Such nucleic acids are referred to hereinafter collectively as the α_{1B} AR transgenes. Preferably the nucleic acid encodes a wild-type or mutant α_{1B} adrenergic receptor. The exogenous wild-type receptor has an amino acid sequence which is different from the amino acid sequence of the α_{1B} adrenergic receptor that is normally found in the animal prior to transformation, i.e., the endogenous α_{1B} adrenergic receptor. The variant receptor is a mutant protein or polypeptide which is derived from a wild-type α_{1B} adrenergic receptor. Variants are produced using techniques which introduce single or multiple amino acid substitutions, deletions, additions or replacements in the wild-type amino acid sequence of an endogenous receptor or an exogenous receptor. Such techniques are well known in the art. The variants may include(a) variants in which one or more, preferably no more than 10, amino acid residues in the wild-type sequence are substituted with conservative or non-conservative amino acids, or (b) variants in which one or more, preferably no more than 10, amino acids are added to the wild-type sequence. Preferred α1B AR transgenes are those which encode a wild-type or mutant hamster, rat or human α1B AR transgene. Preferably, the variant or mutant aclB adrenergic receptor is constitutively active, i.e., the receptor signals even though an agonist is not present. In one embodiment, the α_{1B} AR transgene encodes a mutant U(IB adrenergic receptor, more preferably a mutant hamster α_{1B} adrenergic receptor, in which the amino acid at position 128 is changed from a cysteine to a phenylalanine. (See FIG. 1) In hamsters whose α_{1B} adrenergic receptors have such mutation, the mutant receptor is constantly turned on even when no agonist is present, i.e., the receptor constitutively signals. (See Perez, D. et al., (1996) Molecular Pharmacology 49: 112-122) In another embodiment, the α_{1B} AR transgene encodes a mutant α_{1B} adrenergic receptor, more preferably a mutant hamster α_{1B} adrenergic receptor, in which the cysteine at position 128 is substituted with a phenylalanine, the alanine at position 204 is replaced with a valine, and the alanine at position 293 is replaced with a glutamate. (See FIG. 1). In hamsters whose α_{1B} adrenergic receptors have such mutations, the mutant receptor exhibits robust chronic signaling. The transgenic animal is a non-human mammal, preferably a transgenic rodent, more preferably a transgenic mouse. Such animal is a useful in vivo screening system for drugs that activate, inhibit or reduce activation α_{1B} adrenergic receptors and thereby prevent or alleviate the symptoms associated with neurodegenerative disorders, such as for example Parkinson's disease or epilepsy and cardiovascular disorders such as hypertrophy and hypotension. Transgenic animals which express constitutively active CCIB adrenergic receptors or exogenous wild-type aIB adrenergic receptors on the surface of cells located in the brain are model systems for Parkinson's disease.

A DNA fragment or construct which comprises the α_{1B} AR transgene may be integrated into the genome of the transgenic animal by any standard method such as those described in Hogan et al., “Manipulating the Mouse Embryo”, Cold Spring Harbor Laboratory Press, 1986; Kraemer et al., “Genetic Manipulation of the Early Mammalian Embryo”, Cold Spring harbor Laboratory Press, 1985; Wagner et al., U.S. Pat. No. 4,873,191, Krimpenfort et al U.S. Pat. No. 5,175,384 and Krimpenfort et al., Biotechnology, 9: 88 (1991), all of which are incorporated herein by reference. Preferably, the DNA fragment is microinjected into pronuclei of single cell embryos in non-human mammalian animals, such as mice, rabbits, cats, dogs, or larger domestic or farm animals, such as pigs. These injected embryos are transplanted to the oviduts or uteri of pseudopregnant females from which founder animals are obtained. The founder animals (Fo)founder, are transgenic (heterozygous) and can be mated with non-transgenic animals of the same species to obtain F1 non-transgenic and transgenic offspring at a ratio of 1:1. Alternatively, the Fo transgenics are mated with other Fo transgenic animals to produce F1 transgenic animals that are heterozygous for the transgene (1:1) or homozygous for the transgene (1:4). Preferably, the founder animals are bred with a non-transgenic animal to produce an F1 generation and F2 generation transgenic animals that are heterozygous for the transgene. The heterozygote offspring in the F1 generation or F2 generation exhibit characteristics associated with neurodegenerative disorders. For example, the offspring which are heterozygous for the α_{1B} AR transgene display the symptoms of Parkinson's disease, epilepsies, and cardiovascular disorders. Accordingly, the heterozygous transgenic animals are useful tools for screening agents that block activation of the α1, particularly the α_{1B}, adrenergic receptor.

Thus, the present invention also provides a method for screening agents thought to confer protection against development of neurodegenerative disorders. The method involves treating a transgenic animal of the present invention with the agent and assaying for a reduced incidence or delayed onset of the neurodegenerative disorder as compared to untreated transgenic animals. The indices used preferably are those which can be detected in a live animal such as changes in activity (e.g. horizontal and vertical movements) and locomotion. Additional tests to confirm the effectiveness of the agent by examining pathological changes in the brain or other organs when the animal dies or is sacrificed. Such tests may include histochemical or immunohistochemical examination of targeted tissues. The present invention also provides a method for screening agents thought to improve the symptoms associated with or delay the progression of neurodegenerative disorders such as Parkinson's disease or epilepsy. The method involves treating a transgenic animal of the present invention with the agent of interest and assaying for an improvement, i.e., a reduction in the number or severity and/or a delay in progression of the neurodegenerative symptoms exhibited by such animals as compared to untreated control transgenic animals. Detection of an improvement in the symptoms of the treated animals as compared to the controls indicates that such agent is useful for ameliorating diseases associated with such neurodegenerative disorders.

The wild-type transgenes may be obtained by isolation from genomic sources, by preparation of cDNAs from isolated RNA templates. The variants of such gene may be obtained by site-directed mutagenesis of a cDNA or RNA which encode the wild-type UIB adrenergic receptor.

The α_{1B} AR transgene is operably linked to a promoter that is used to increase, regulate, or designate to certain tissues expression of the transgene. The promoter may be from a heterologous source, i.e., it is a promoter which is not naturally associated with the nucleic acid. Included among heterologous promoters are those from a different species or a different gene. The promoter may be ubiquitous, i.e. it drives expression of the transgene in the cells or organs throughout the body of the transgenic animal. Alternatively, the promoter may be tissue specific, i.e. it regulates expression of the operably-linked transgene in specific cells or tissues, e.g. neurons. The promoter may be a constitutive or an inducible promoter. Preferably the promoter is a tissue specific promoter which drives localized expression of the transgene on the surface of cells in all sympathetically innervated tissues, including but not limited to neurons and smooth muscle cells. In a transgenic mouse, a highly preferred promoter is the mouse α_{1B} AR gene promoter which drives endogenous tissue distribution of the α_{1B} AR transgenes.

The present invention also provides a method of treating the symptoms of neurodegenerative disorders in a subject, particularly those neurodegenerative disorders which involve locomotor impairment and/or seizures. As used herein the term subject, refers to a mammalian animal, preferably a human. By “treating” is meant ameliorating or tempering the severity of the disorder or the symptoms associated therewith. In cases of such as for example Parkinson's disease, the pharmaceutical composition is administered either when patients have clinical symptoms, or when a genetic mutation is identified. Preferably, the protocol involves oral administration of a pill or water soluble mixture, or injection, preferably intravenous injection. In the case of neurodegenerative disorders that involve epileptic seizures, the pharmaceutical composition is administered when the patient shows clinical signs of seizure disorders, such as a cortical dysfunction. The protocol involves oral administration of the pharmaceutical composition, which preferably is in the form of a pill or water soluble mixture, or injection of the pharmaceutical composition, preferably intravenous injection.

Pharmaceutical Composition

The pharmaceutical composition comprises a biologically effective amount of an α1 or α_{1B} adrenergic receptor antagonist, and preferably a relatively inert topical carrier. Many such carriers are routinely used and can be identified by reference to pharmaceutical texts. Examples of known α1 AR antagonists are terazosin, which is sold under the tradename Hytrin and currently used for the treatment of benign prostatic hypertrophy and phentolamine, which is currently used in the treatment of high blood pressure and erectile dysfunction. Other α1 AR antagonists are prazosin, 5 methylurapidil, WB 4101, niguldipine, HEAT, indoramine, coryanthine, spierone, benoxathian, spiroxatrine, and chloroethylclonidined.

Carrier

The acceptable carrier is a physiologically acceptable diluent or adjuvant. The term physiologically acceptable means a non-toxic material that does not interfere with the effectiveness of the antagonist. The characteristics of the carrier will depend on the route of administration and particular compound or combination of compounds in the composition. Preparation of such formulations is within the level of skill in the art. The composition may further contain other agents which either enhance the activity of the antagonist or complement its activity. The composition may further comprise fillers, salts, buffers, stabilizers, solubilizers, and other materials well known in the art.

Dosage

A biologically effective amount is an amount sufficient to partially or completely relieve the symptoms associated with the neurodegenerative disorder. The effective amount can be achieved by one administration of the composition. Alternatively, the effective amount is achieved by multiple administration of the composition to the subject.

The present invention will be described in greater detail with the aid of the following examples which should be considered as illustrative and non-limiting.

### EXAMPLE 1

α_{1B} AR Transgene Constructs.

Plasmids comprising a cDNA encoding the wild-type hamster a1B adrenergic receptor, a constitutively active single mutant hamster a1B adrenergic receptor, and a constitutively active triple mutant α_{1B} adrenergic receptor operably linked to the mouse isogenic α_{1B} AR promoter were prepared using standard techniques. The single mutant α_{1B} AR cDNA was prepared by site-directed mutagenesis of wild-type hamster α_{1B} AR cDNAas described in Perez et al. (1996) Molecular Pharmacology 49:112-122, which is specifically incorporated herein by reference. The triple mutant α_{1B} AR cDNA was prepared by site-directed mutagenesis of wild-type hamster α_{1B} AR cDNA as described in J. Hwa et al., Biochem. 36, 633 (1997), which is specifically incorporated herein by reference. The single mutant, A C128F(S) and the triple mutant, C128F/A204V/A293E (T), have both been shown to spontaneously couple to G_{q}. and to thereby increase signaling.

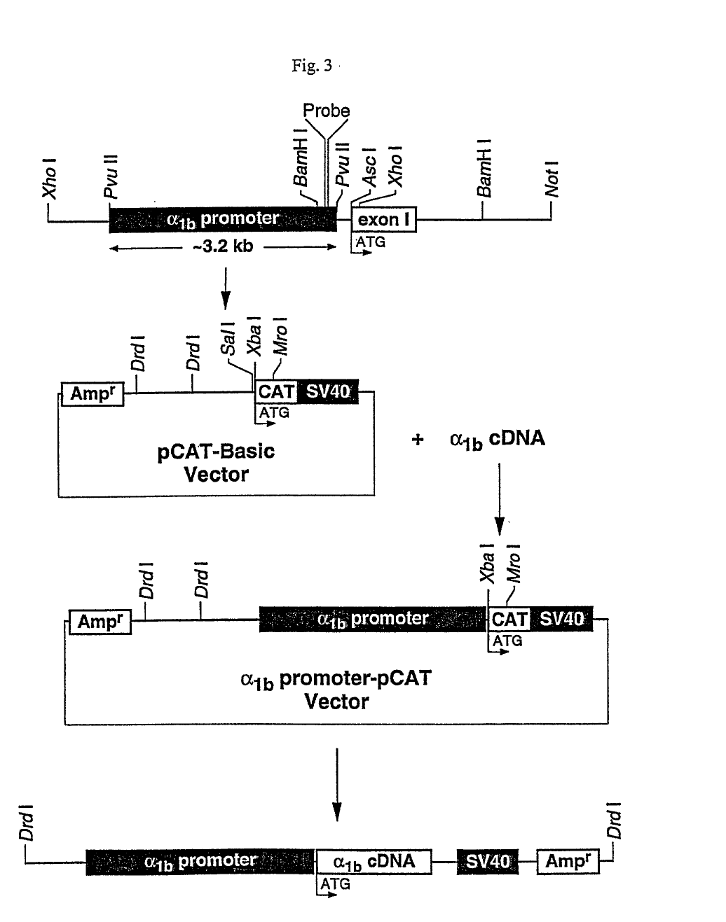

Promoter sequence from the murine α_{1B}AR gene was isolated from a mouse genomic library (129SVJ female liver, Stratagene, La Jolla, Calif.) via plaque hybridization screening [Zuscik et al., Mol. Pharm. 56, 1288 (1999)]. A 3.4 kb promoter fragment was subcloned into the SalI site of the pCAT basic vector (Promega Biotech, Madison, Wis.) and in vitro functional fidelity was confirmed [Zuscik et al., Mol. Pharm. 56, 1288 (1999)]. Subsequently, three separate transgenes were constructed from this α_{1B}AR promoter-pCAT scaffold. (See FIG. 3) First, the CAT gene reading frame was disrupted via an XbaIMroI digest of the plasmid to remove 258 bp of the CAT gene including its start site. After flushing of this CAT-less plasmid with Klenow, WT-[S. Cotecchia et al., Proc. Natl. Acad. Sci. USA 85, 7159 (1988)], C 12817-[D. M. Perez et al., Mol. Pharm. 49, 112 (1996)], and C128F/A293E/A204V-α_{1B}AR [J. Hwa et al., Biochem. 36, 633 (1997)] cDNAs were blunt end subcloned into the former CAT site, immediately 3′ of the 3.4 kb α_{1B}AR promoter. Correct orientation of the cDNAs and the presence of the appropriate mutations was confirmed by sequencing. Large-scale preparations of plasmid DNA for each transgene were purified using a kit (Wizard Maxipreps, Promega, Madison, Wis. An antibody tag , specifically an identification epitope known as ID4 epitope tag was engineered on the 3′ end of the OCIB AR transgenes. Such epitope is useful for detection of the receptor protein in individual organ systems.

### EXAMPLE 2

Transgenic Mice Comprising an α_{1B} AR Transgene

Approximately 200 copies of each linearized transgene were injected into the pronuclei of individual one cell B₆/CBA mouse embryos according to methods described in Hogan, B. et al, (1986) Manipulating the Mouse Embryo: A Laboratory Manual. Cold Spring Harbor Laboratory, Cold Spring Harbor, N.Y., Sections C and D, pp81-297, which is specifically incorporated herein by reference. The surviving microinjected eggs were then transplanted into the oviduct of 0.5 day pseudopregnant females (outbred Swiss Webster strain), which had been previously mated to vasectomized males. Approximately, 20-30 eggs were transferred to each foster mother, which produced an average litter size of 4 pups. Genomic DNA was isolated from −5 mm tail sample obtained from 10 day old pups and the presence of the transgenic construct determined by Southern blot analysis. Probes were comprised of either a 600 bp BanHI-XhoI fragment proximal to the ATG codon of the hamster α_{1B}AR cDNA (α_{1B}AR probe) or the 1392 by of sequence between the EcoRI sites that encompass the SV40 domain of the transgene (SV40 probe). Probes were labeled with [α-³²P]dCTP (New England Nuclear, Boston, Mass.) using a kit (Random Primed Labeling Kit, Boehringer Mannheim, Indianapolis, Ind.). Of the 11 original founders, 1 WT, 2 single mutant and 1 triple mutant founder did not transmit the gene to subsequent generations.

Expression of the Transgene Proteins

The distribution and magnitude of transgene protein expression in F1 and F2 generation heterozygous mice was then determined via saturation binding analysis of membranes prepared from skeletal muscle, tongue, liver, heart, lung, brain, kidney and spleen.

Membranes used in binding assays were prepared from various tissues as follows. Tissues were placed in ice cold buffer A (0.2 to 0.3 mg/ml final) composed of 10 mM hepes (pH 7.4), 250 mM sucrose, 5 mM EGTA, 12.5 mM MgCl₂ and a cocktail of protease inhibitors. Tissues were disrupted for 30 sec with a polytron, transferred to a dounce homogenizer, diluted 1:7 in buffer A, and homogenized 10× with each a loose and tight pestle. Homogenates were spun for 5 min at 300 g to remove fat and for 5 min at 1250 g to remove nuclei. Homogenates were spun for 15 min at 35,000 g and pellets were resuspended in ice cold buffer B composed of 20 mM hepes (pH 7.4), 100 mM NaCl, 5 mM EGTA 12.5 MM MgCl₂ and a cocktail of protease inhibitors. This spin/resuspension was repeated twice. After resuspension in buffer B containing 10% glycerol, the final pellet was homogenized, analyzed for protein concentration by Bradford and frozen at −70° C. at a final concentration of less than 5 mg/ml. Heart, skeletal muscle and tongue homogenates were treated similarly except prior to the first 35,000 g spin, tissues were incubated for 15 min at 4° C. in an equal volume of 0.5 M KCI. Liver homogenates were also treated similarly except after douncing, tissue was spun at 1 5,000 g for 20 mice and the pellets were resuspended in 70% buffer A/30% Percoll. Samples were spun for 1 hr at 35,000 g and the intermediate layer between the red cells and lipids was harvested. The harvested layer was diluted 1:4 in ice cold buffer B and subsequent 35,000 g spin/wash steps were followed as described.

Saturation binding was performed as in [D. M. Perez et al., Mol. Pharm. 49, 112 (1996)] using the non-selective α₁AR antagonist [¹²⁵I]HEAT as the radiolabel. Reactions contained 20 mM hepes (pH 7.5), 1.4 mM EGTA, 12.5 mM MgCl₂, membranes, 10 μM phentolamine to block non-specific binding and increasing concentrations of [²⁵I]HEAT ranging from 25 to 2000 pM. Reaction mixtures were incubated for 1 hr at 22° C., stopped by addition of cold hepes buffer, and filtered onto glass fiber filters using a Brandel cell harvester.

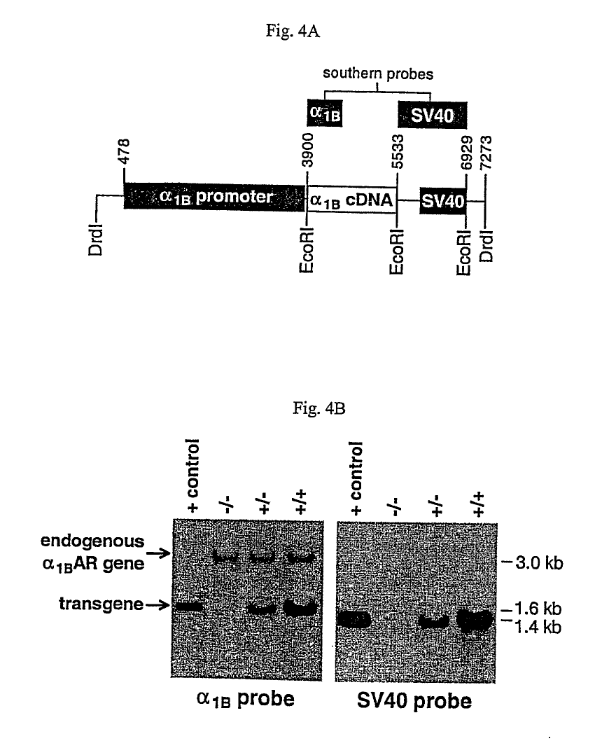

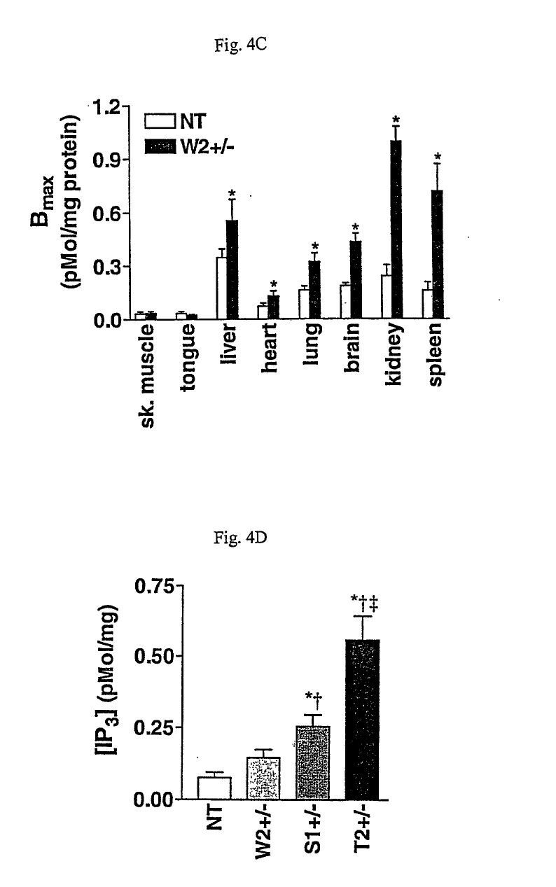

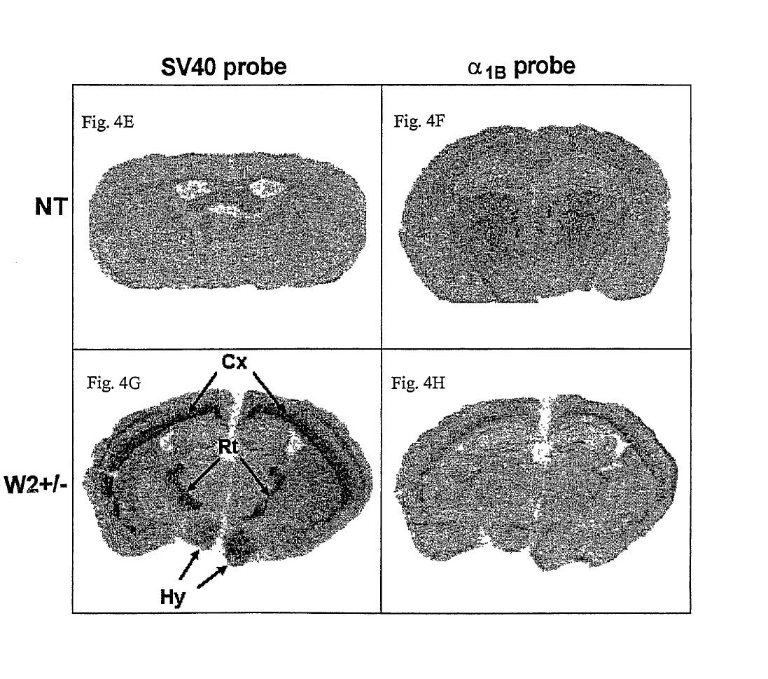

FIG. 4Cshows the distribution and magnitude of expression in non-transgenic (NT) and W2+/− mice. As expected, α_{1B}AR-negative skeletal muscle and tongue showed equally low Bmax values in NT and W2+/− animals. However, in α_{1B}AR-positive liver, heart, lung, brain, kidney and spleen, W2+/− mice showed significant increases in Bₘₐₓ over NT controls. Distribution and magnitude of receptor overexpression seen in W2+/− mice was not significantly different from that seen in W1+/−, S1+/−, T1+/− and T2+/− mice

To confirm constitutive signaling of these overexpressed receptors in the transgenic lines; inositol-1,4,5-trisphosphate (IP₃) levels were determined in livers from 6 month old NT, W2+/−, S I+/− and T2+/− mice using a commercially available radio-receptor assay kit (New England Nuclear, Boston, Mass.). Livers were minced with a scalpel and incubated for 1 hr with gentle agitation at 37° C. in 25 ml serum-free DMEM containing 10 mM LiCl₂. IP₃ was extracted using trichloroacetic acid and quantitated by competition binding using [³H]IP₃ according to the kit's instructions. As shown in FIG. 4D, IP3 levels are significantly higher in livers from S1+/− and T2+/− mice than in livers from age-matched NT mice. The rank order increase in IP₃ pool size seen between the various lines (T2>S1>W2) coincides with the strength of constitutive signaling that was found for these receptors in vitro (4,5).

To investigate the endogenous localization of the α_{1B}AR in the brain and to confirm fidelity of transgene localization, in situ mRNA hybridization analysis was performed on coronal brain sections from NT and W2+/− mice. In the NT brain, a riboprobe specific for the transgene-unique SV40 transcript showed no specific hybridization signal (FIG. 4E), while a riboprobe specific for the hamster α_{1B} transcript identified mRNA expressed from the endogenous α_{1B}AR gene. (FIG. 4F). Comparatively, the SV40 riboprobe showed a robust signal in a W2+/+ brain, indicating transgenic expression of SV40 mRNA. These SV40 transcripts were localized to discreet regions of the basal forebrain and diencephalon, including the reticular thalamic nucleus and portions of the hypothalamus (FIG. 4G). A similar pattern of expression was detected with the hamster α_{1B} probe (FIG. 4H). Based on the general regional overlap of transgene expression with endogenous alBAR expression, these results indicate promoter fidelity.

It is believed that overexpression of the WT receptor induces a significant increase in the number of receptors spontaneously isomerized to the active conformation (R*), thus causing increased G protein coupling and signaling. Increased coupling and signaling in various organs also results by expressing the constitutively active mutant forms of the receptor that achieve the R* conformation even without agonist binding.

Phenotype of the Transgenic Animals

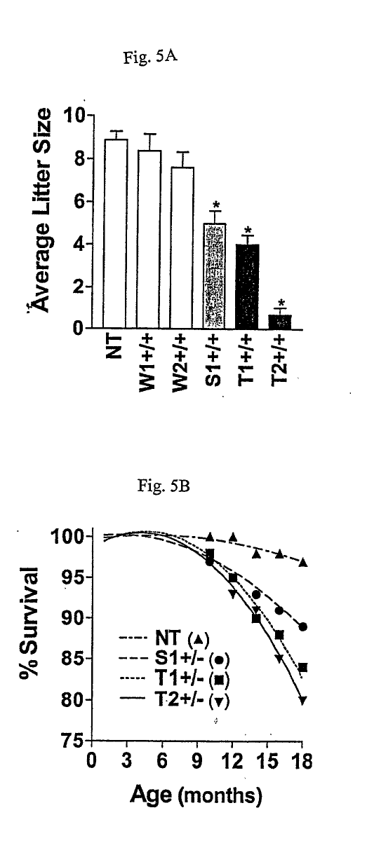

At birth, heterozygous mice overexpressing WT or expressing constitutively active mutant α_{1B}ARs were viable and showed no gross phenotypic abnormalities. Older heterozygous mice (>12 months) were generally characterized to possess somewhat reduced longevity and significantly lower body weight. Compared to 97% survival of NT mice at 18 months, survival rates for S1+/−, T1+/− and T2+/− mice were between 80% and 90% (FIG. 5B). Longevity of W1+/− and W2+/− mice was not different from the NT control. Additionally, at 14 months, W2+/−, S1+/−, T1+/− and T2+/− lines exhibited reduced body weight compared to age-matched NT controls (FIG. 5C). This phenotype did not arise in mice less than 6 months of age.

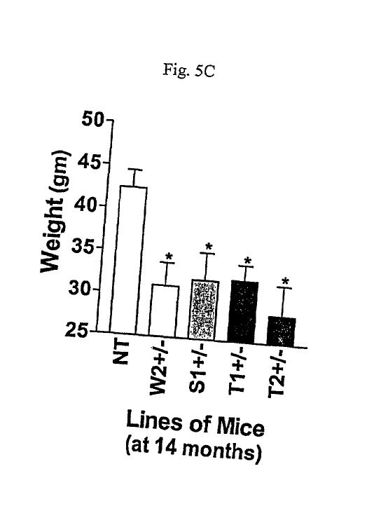

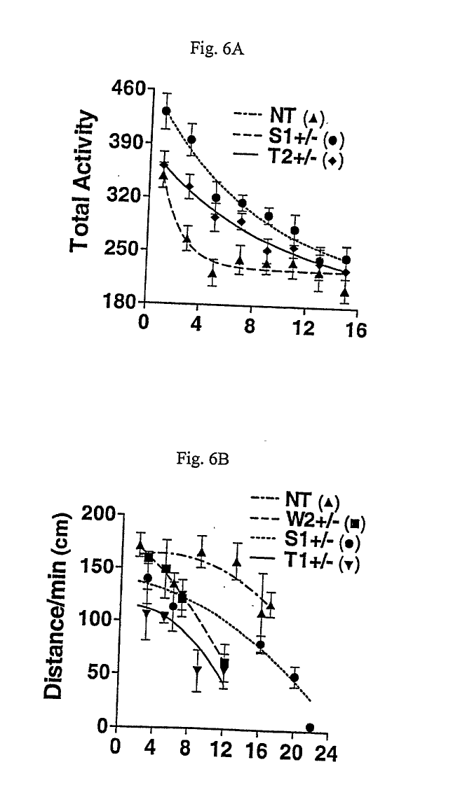

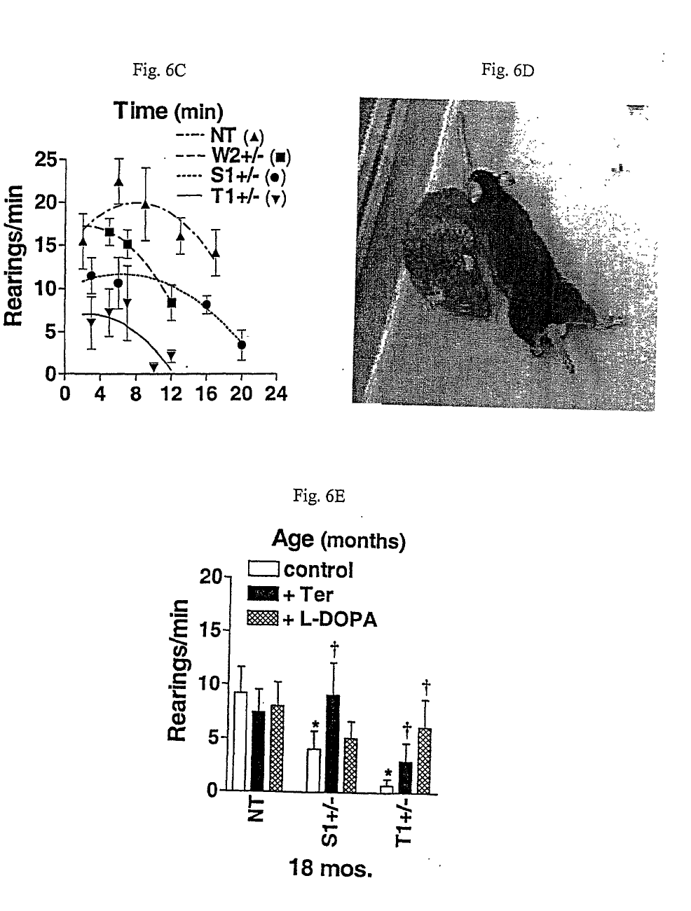

Despite a lack of obvious gross abnormalities, a striking behavioral phenotype was observed in transgenic mice less than 2 months of age. Following exposure of these youngest mice to open field stress, significant increases in total activity relative to NT mice were observed (FIG. 6A). Compared to age-matched NT controls, S1+/− mice not only displayed more total activity upon initial exposure to the open field, but also showed a significant delay in recovery to basal activity. T2+/+ mice, which did not show initial increased activity relative to NT controls, showed a delayed recovery to basal activity which was similar to that seen in the S 1+/− group. W 1+/− and W2+/− mice did not exhibit hyperactivity.

In older mice (>2 months), where constitutive receptor function has induced a more “chronic” UXBAR stimulation, the hyperactive phenotype was completely lost and a severe hindlimb locomotor dysfunction emerged. This phenotype was characterized by an age- progressive loss of horizontal ambulation (FIG. 6B) and a decrease in the number of times the mice reared up onto their hindlimbs (FIG. 6C). The most severely impaired animals exhibited abnormal gait with hind paws flattened to the ground, sprawling and dragging of the hindlimbs, a lack of spontaneous locomotion and a prevalence of tremor (FIG. 6D).

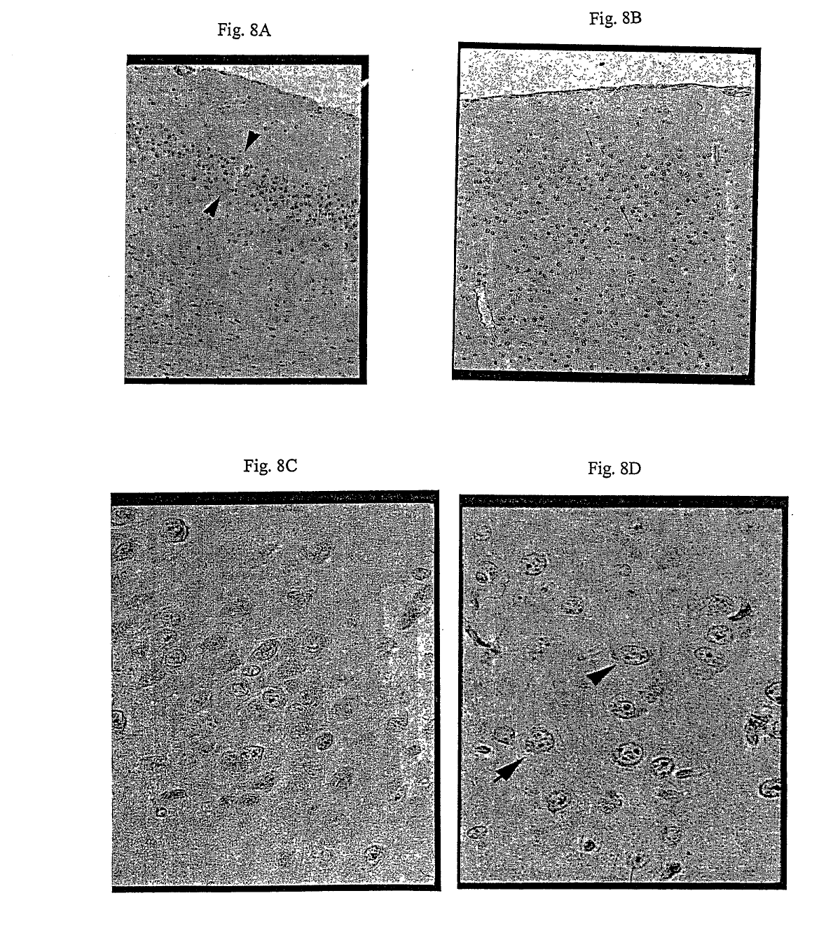

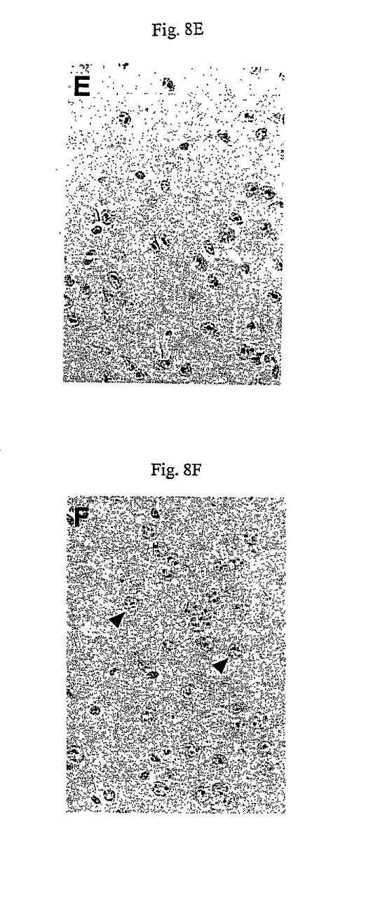

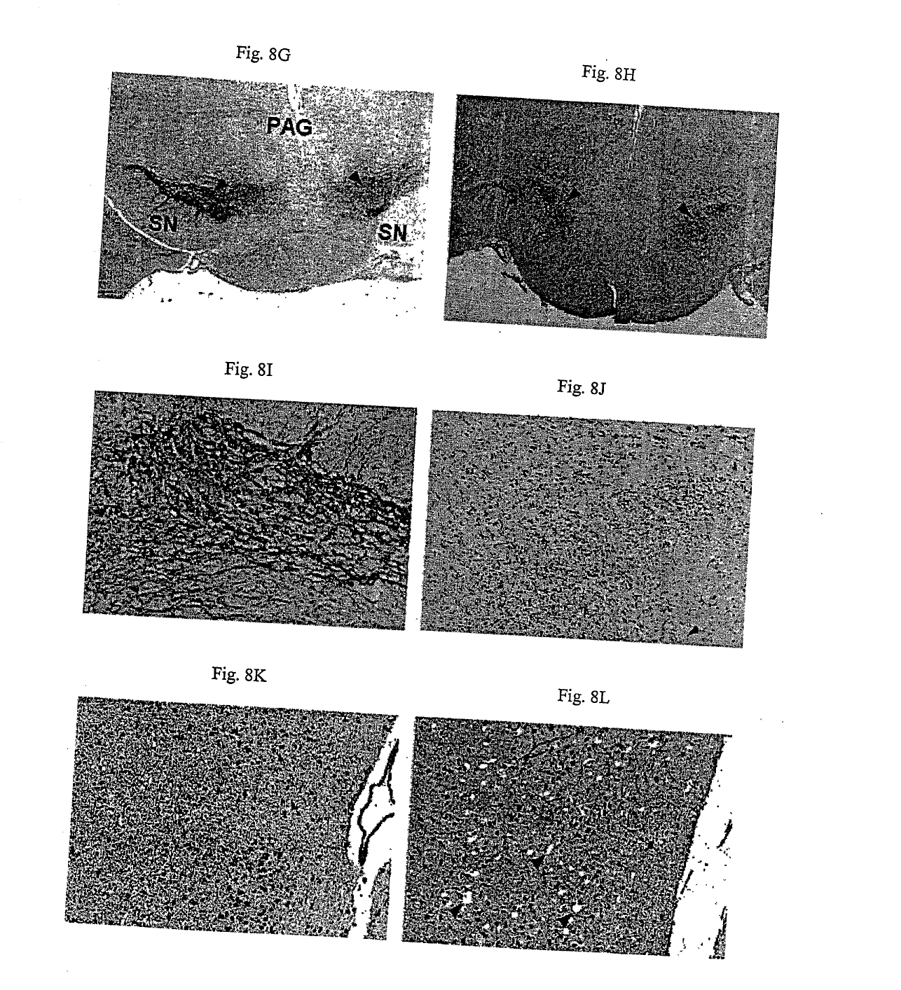

A reduction of tyrosine hydroxylase (TH) content in striatal tracts is a prominent feature in Parkinson's disease. In fact, since TH catalyzes the formation of L-DOPA, the rate limiting step in the formation of dopamine, Parkinson's disease has been regarded as a TH-deficiency syndrome. Consistent with this neurodegenerative marker in Parkinson's disease, 11 month old T2+/− mice showed significant loss of TH immunoreactivity in the substantia nigra (FIGS. 8H and 8J) compared to age matched NT mice (FIGS. 8G and 8I). Higher power magnification shows a loss of neuronal-cell bodies and axonal projections. Also indicative of a Parkinsonian-like syndrome in α_{1B} AR overactive mice was a net neuronal loss in the periaqueductal gray area. Prevalence of vacuolar dead space seen in the periaqueductal gray of 11 month T2+/− mice (FIG. 8K) relative to age-matched NT mice (FIG. 8K) is indicative of this neuronal loss, and further implicates the α_{1B}AR system in Parkinsonian-like neurodegeneration. Overall, these neurodegenerative findings are consistent with the locomotor phenotypes seen in α_{1B}AR transgenic mice.

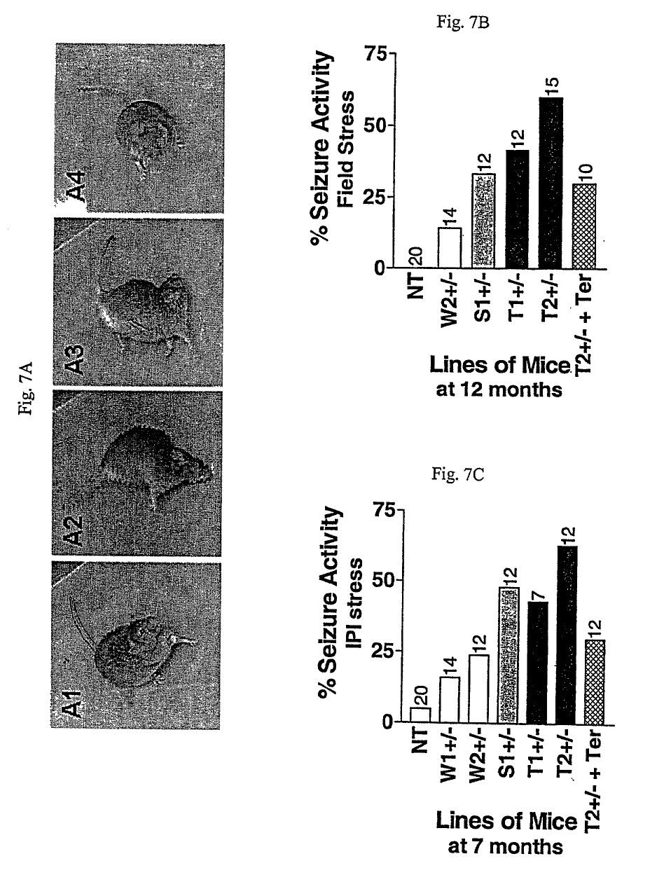

In addition to locomotor impairment, older mice possessing systemic α_{1B}AR overactivity also exhibited grand mal-type seizures. The seizure event seen in W2+/−, S 1+/−, T1+/− and T2+/− mice at twelve months of age was evoked by exposure to the open field (i.e. handling-invoked stress) and was characterized by behavioral arrest, facial twitching, loss of balance, forelimb automatism, whole body jerking and hypersalivation. Seizure duration was typically 30 sec, with a full recovery usually achieved within 2 min. Age-matched NT mice did not manifest the seizure phenotype. Serial snap shots of the seizure event in a T2+/− mouse are shown in FIG. 7A and quantification of percent seizure activity induced by the open field seen in various lines is shown in FIG. 7B. The percentage of mice exhibiting seizures correlated with the level of α_{1B}AR overstimulation (i.e. triple mutant>single mutant>WT.

Seven month old transgenic mice, which did not display the seizure phenotype upon exposure to the open field, did manifest seizures upon intraperitoneal injection of 50 μl sterile saline (FIG. 7C). Seizures seen in response to this intraperitoneal injection stress (IPI stress) resembled the open field stress-induced event in terms of severity, characteristics and duration. As with older T2+/− mice exposed to the open field, terazosin reduced the number of younger T2+/− mice displaying IPI-induced seizures. Suggesting that the manifestation and severity of the seizure phenotype was age-progressive, transgenic mice (from any line) younger than 5 months of age that were exposed to open field or IPI stress did not display seizure activity. This age-progressive nature of the phenotype is also borne out by the finding that open field stress alone was not adequate to induce seizures in seven month old mice. Rather, a higher level of stress input (i.e. injection) was required to induce the phenotype. In conjunction with the aforementioned involvement of the α_{1B}AR in locomotor activity, the manifestation of seizures in these mice suggests a novel involvement of this receptor in neuronal network excitability.

Brain tissue from subjects afflicted with neurodegenerative disease can be histologically distinguished from normal brain tissue by the presence of markers specific for ongoing reactive gliosis. Reactive gliosis, which is present in regions experiencing neuronal damage and/or death, involves an infiltration of reactive astrocytes which partially facilitate the repair process. These reactive astrocytes are histologically distinct due to their swollen morphology and prominent nucleoli. Indicating neurodegeneration in the α_{1B}BAR overactive mouse at 10 months of age, hematoxylin/eosin stained coronal sections of W2+/− brains showed disorganization of cortical laminae (FIG. 8B) relative to the intact laminar organization seen in age-matched NT brains (FIG. 8A). Higher power magnification of these same cortical sections also showed loss of neuronal-type cells with a significant infiltration of a reactive astrocytic cell type in W2+/brains (FIG. 8D) that was absent in NT brains (FIG. 8C). Hypothalamic regions of T2+/brains exhibited a similar loss of neurons with an increase in astrocytic infiltration (FIG. 8F) relative to analogous sections from the age matched NT control (FIG. 8E).

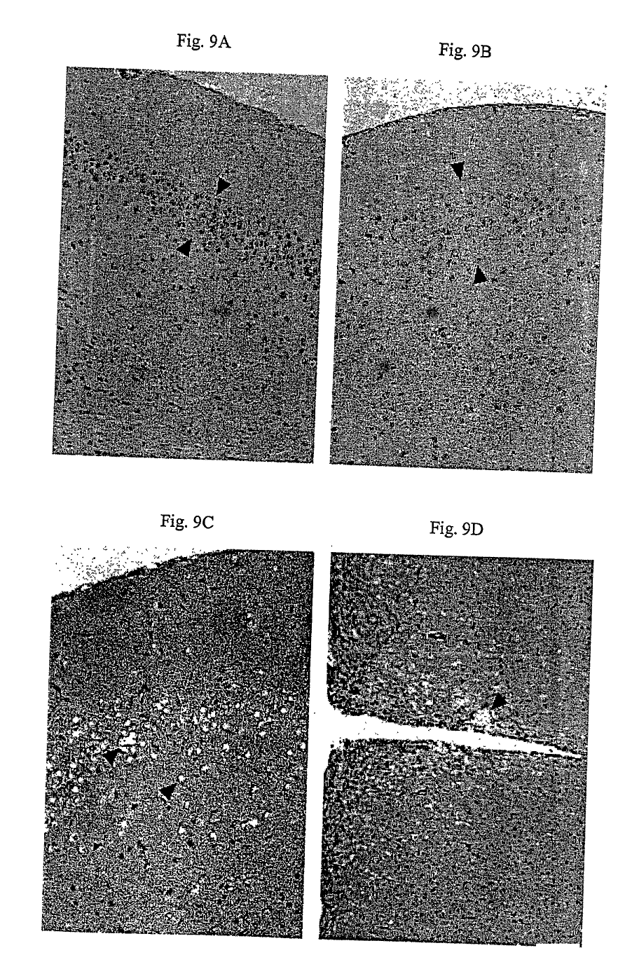

The T2 mice, which showed the most severe seizure phenotype also exhibited significant neurodegeneration as evidenced by vacular dead space throughout the cortex (FIG. 9C) as well as the hypothalamus (FIG. 9D). This degeneration was seen in foci, i.e. it was concentrated only in certain depths of the brain. Such changes are consistent with a grand mal seizure disorder.

Peripheral Effects in the Cardiovasculature

The transgenic mice displayed significant cardiac hypertrophy as indicated by an elevated heart to body weight ratio (FIG. 11) as well as by echocardiographic analysis. This analysis indicated significant increases in heart muscle and wall thickness such as in the interventricular septum diameter (IVS) in S 1 and T2 mice as well as the posterior wall dimension (PWd). Indicative of diastolic dysfunction and muscle stiffness, the isovolumetric relaxation time (IVRT) was increased in both S1 and T2 mice indicating that it took the heart of these animals a longer time to relax. The heart rate in all transgenic lines was substantially decreased from nontransgenic controls. The hypertrophy could not be due to pressure-induced effects since the pressure phenotype in these mice are hypotensive.

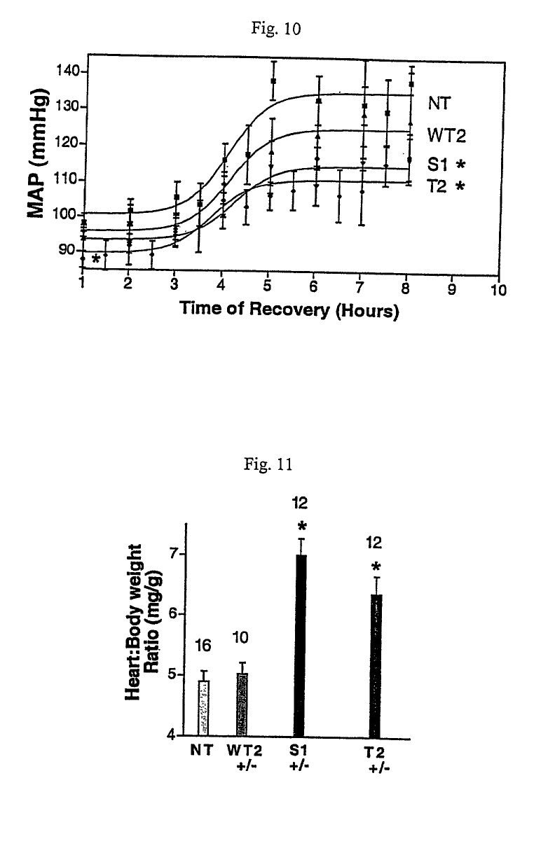

Since α₁-subtypes are known to be a major regulator of blood pressure by their localization and controlling contraction of the arterials, blood pressure was analyzed by two invasive methods. In the first, the carotid artery was cannulated and basal pressure recorded in the conscious and unrestrained mouse. After 8 hours of recovery when the mice are fully moving, both the S1 and T2 mice display basal hypotension.(FIG. 10A) This result was confirmed in separate studies in which the femoral artery was cannulated and the blood pressure measured in response to an α₁ pressor agent, phenylephrine, given under anesthesia. As shown in FIG. 10B, the S1 mice displayed both a basal depression in pressure as well as an impaired response to phenylephrine.

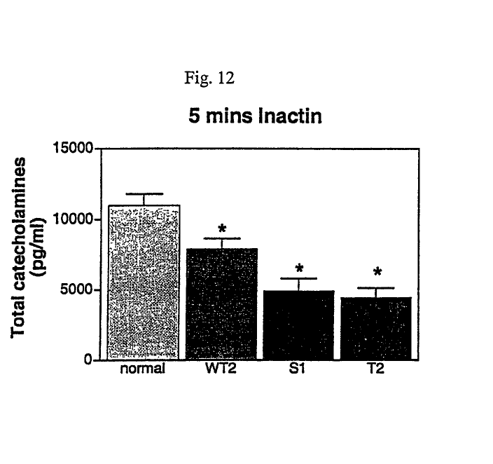

Since both basal and pressor responses of the resistance arteries seemed impaired in the transgenics, we next explored whether sympathetic tone could be responsible for the hypotension. Plasma catecholamines, epinephrine and norepinephrine, were determined via radioenzymatic assay by the laboratory medicine department of the Cleveland Clinic. As shown in FIG. 12, total catecholamines were reduced significantly in all three transgenic lines suggesting a decrease in sympathetic output.

TABLE 1
Echocardiographic Analysis of Nontransgenic and Transgenic mice

| | | | | | | IVRTmse | |
|---|---|---|---|---|---|---|---|
| | Weight | LA/wt | IVS/wt | PWd/wt | F S% | c | HR |
| Normals | 38.1 = 1.7 | 0.039 ± | 0.031 ± | 0.026 ± | 64.5 ± 1.1 | 15.7 ± 1.1 | 573 ± 53 |
| | | .001 | .002 | .001 | | | |
| WT2 | 23.5.1 ± | 0.074 ± .2 | 0.037 ± | 0.034 ± | 48.9 ± 6.2 | 25.5 ± 1.7 | 344 ± 42 |
| | 2.1* | | .002 | .001 | | | * |
| S1 | 31.7 ± 0.8* | 0.055 ± | 0.040 ± .001 | 0.034 ± | 54.5 ± 4.9 | 29.1 ± 3.1 | 350 ± 19 |
| | | .003 | * | .001 | | * | * |
| T2 | 33.4 ± 2 | 0.051 ± | 0.041 ± | 0.038 ± .003 | 55.1 ± 4.6 | 27 ± 3.2* | 407 ± 43 |
| | | .003 | .002* | * | | | * |

### EXAMPLE 3

Treatment of Animals Exhibiting Symptoms of Neurodegenerative Disorders with α_{1B} Adrenergic Receptor Antagonists

Receptor overactivity was inhibited in S1+/− and T1+/− mice via treatment with the α_{1B}AR-specific antagonist terazosin. Animals were treated with the drug at a target dose of 0.05 mg/Kg of body weight/day via the drinking water. After four weeks of treatment, a partial rescue of the rearing behavior was observed (FIG. 6E), with the more dramatic improvement seen in S1+/− mice possibly being due to the less severe symptoms present in these animals. In addition, administration of L-DOPA (in the form of Sinemet), which is the prevailing drug used in the treatment of Parkinson's disorder, dramatically reversed the rearing deficit in the most severely -affected mice (T1). (FIG. 6E). In contrast L-DOPA treatment of the S1 animals, which exhibit less neurodegeneration than the T1 animals, did not improve the locomotor activity of these animals. These results indicate the cc,-AR antagonists may be useful in ameliorating the symptoms of Parkinson's disease, particularly as an early treatment, i.e. when symptoms of the disorder begin to manifest.

T2 mice at 7 months and 12 months of age were treated with the α_{1B} AR antagonist terazosin at a target dose of 0.05 mg/Kg body weight/day via the drinking water. At four weeks of treatment, the percent seizure activity in the treated mice was determined and compared to control T2 mice which did not receive the antagonist (FIG. 7B). The seizure event was partially reversible i.e., fewer events in the treated T2 mice at 12 months of age. In younger mice, i.e. mice at 7 months of age, that were induced to have seizure via an IPI stress, 4 weeks of treatment with the antagonist partially reversed the phenotype FIG. 7C These results indicate oxl antagonists may be useful in treatment of seizures, particularly epilepsies or other types of seizure invoked by cortical disruption or degeneration.

All publications mentioned in this specification are herein incorporated by reference to the same extent as if each individual publication was specifically and individually indicated to be incorporated by reference.

The invention now being fully described, it will be apparent to one of ordinary skill in the art that many changes and modifications can be made thereto without departing from the spirit or scope of the appended claims.

What is claimed is:

1. A transgenic non-human mammalian animal having integrated within its genome a transgene encoding an exogenous wild-type α_{1A}, α_{1B}, or α_{1D} adrenergic receptor or a transgene encoding a constitutively-active mutant α_{1A}, α_{1B}, or α_{1D} adrenergic receptor, wherein the transgene is operably linked to a promoter that drives expression of the transgene in cells innervated by the sympathetic nervous system, and wherein the transgenic animal exhibits an abnormal phenotype.

2. The transgenic animal of claim 1 wherein the transgene encodes an exogenous wild-type α_{1B} adrenergic receptor or a constitutively active mutant α_{1B} adrenergic receptor.

3. The transgenic animal of claim 1 wherein the animal is a mouse and exhibits a neurodegenerative disorder-type phenotype.

4. The transgenic animal of claim 1 wherein the animal is a mouse and exhibits a phenotype resembling a cardiovascular disease.

5. The transgenic animal of claim 1 wherein the promoter is the promoter of the animal's endogenous α_{1B} adrenergic receptor.

6. The transgenic animals of claim 1 wherein the transgene encodes a constitutively active mutant hamster, rat, or human α_{1B} adrenergic receptor.

7. The transgenic animal of claim 1 wherein expression of the transgene results in the animal exhibiting Parkinson's disorder type symptoms.

8. The transgenic of animal of claim 1 wherein the transgene encoding a signal peptide.

9. A method of screening for a compound which modulates function of α_{1B} adrenergic receptor comprising:
administering the compound to the transgenic animal of claim 1; and
assaying for changes in the abnormal phenotype of said animal.

10. The method of claim 9 wherein the animal exhibits neurodegenerative symptoms and wherein the assay involves assaying for an improvement in or a delay in progression of the symptoms.

11. The method of claim 9 wherein the animal exhibits symptoms of a cardiovascular disorder and wherein the assay involves assaying for an improvement in or delay in progression of the symptoms.

12. The method of claim 9 wherein the assay involves evaluating the locomotor activity of the animal.

13. The method of claim 9 wherein the animal exhibits seizure type symptoms and wherein said assay involves evaluating the effect of the compound on the frequency, severity, or duration of said seizures.

14. A method of screening a drug for activity against a neurodegenerative disorder or a cardiovascular disorder, comprising
administering the drug to a transgenic mouse whose somatic cells comprise a transgene encoding an exogenous wild-type α_{1B} adrenergic receptor or a transgene encoding a constitutively-active mutant α_{1B} adrenergic receptor, wherein the transgene is operably linked to a promoter that drives expression of the transgene in cells innervated by the sympathetic nervous system, and wherein the transgenic animal exhibits symptoms characteristic of a disorder selected from the group consisting of a neurodegenerative disorder, a cardiovascular disorder, and a combination of a neurodegenerative and a cardiovascular disorder; and
monitoring the mouse for the effects of said drug on said symptoms.

15. The method of claim 14 wherein the transgenic mouse overexpresses an exogenous α_{1B} adrenergic receptor on the surface of cells in the brain of said animal.

16. The method of claim 14 wherein the transgenic mouse expresses a constitutively active mutant α_{1B} adrenergic receptor on the surface of cells in the brain of said animal.

17. A method for treating a subject with a neurodegenerative disorder, comprising:
administering to said subject a biologically effective amount of a compound capable of blocking activation of α₁ adrenergic receptors

18. The method of claim 17 wherein said compound is an α_{1B} adrenergic receptor antagonist.

19. The method of claim 17 wherein said subject has exhibited symptoms characteristic of Parkinson's disease.

20. The method of claim 17 wherein the subject has exhibited seizures.

21. The method of claim 17 wherein the subject has exhibited locomoter impairment.
